(19) United States
(12) Patent Application Publication
(10) Pub. No.: US 2024/0198076 A1
(43) Pub. Date: Jun. 20, 2024

(54) Detectable Disinfecting Cap and Method for Tracking Thereof

(71) Applicant: Becton, Dickinson and Company, Franklin Lakes, NJ (US)

(72) Inventors: Ryan Fernandez, Nutley, NJ (US); Zachary Traina, Verona, NJ (US); Joseph David Mummert, Ramsey, NJ (US); Yashar Seyed Vahedein, Mountain View, CA (US); Mark Jesser, Wyckoff, NJ (US); Erik Kurt Witt, Oakland, NJ (US); Dylan Gregory DeBoer, Vernon Hills, IL (US)

(21) Appl. No.: 18/068,049

(22) Filed: Dec. 19, 2022

(51) Int. Cl. A61M 39/20; A61M 39/16

(52) U.S. Cl. CPC A61M 39/20; A61M 39/16

(57) ABSTRACT

A disinfecting cap engageable with a catheter connection includes a housing defining an inner cavity, with the housing having a top wall, a multi-sided sidewall structure extending down from the top wall and including a plurality of sidewall sections, and an open bottom end. The disinfecting cap also includes a disinfecting member configured to disinfect portions of the catheter connection. Each of the plurality of sidewall sections of the housing includes a fiducial marker thereon, the fiducial marker including a unique identifier for the disinfecting cap.

### FIELD OF THE INVENTION

The present disclosure relates generally to caps for medical connectors and, in particular, to detectable disinfecting caps that include fiducial markers thereon that enable tracking and identification thereof as a part of a vascular access management system.

### DESCRIPTION OF RELATED ART

Catheters are commonly used to administer fluids into and out of the body. Patients in a variety of settings, including in hospitals and in home care, receive fluids, pharmaceuticals, and blood products via a vascular access device (VAD) that includes such a catheter inserted into a patient's vascular system. A common VAD includes a plastic catheter that is inserted into a patient's vein, with a length of the catheter varying from a few centimeters when the VAD is a peripheral intravenous catheter (PIVC) to many centimeters when the VAD is a central venous catheter (CVC), as examples. A VAD may be indwelling for short term (days), moderate term (weeks), or long term (months to years).

If not properly maintained or if exposed to a non-sterile environment, a VAD can become contaminated, sealed with blood clots, and/or can spread infection. Further, bacteria and other microorganisms may gain entry into a patient's vascular system from access hubs, ports, or valves upon connection to the VAD to deliver a fluid or pharmaceutical to a patient. Therefore, each access hub/port/valve of a VAD that is configured for attachment to a VAD is associated with some risk of transmitting a catheter related bloodstream infection (CRBSI) to a patient.

In order to decrease CRBSI cases and to ensure VADs are used and maintained correctly, many medical facilities implement sterile practices and protocols to ensure that VADs and access connectors are used properly and do not become sealed or infected. These protocols often include sterilizing the access hubs, ports, and VADs, including requiring that access hubs, ports, and valves be covered with disinfecting caps when not in use, to prevent microbial ingress into the hub, port, or valve and to sterilize areas of the hub, port, or valve of the VAD. Disinfecting caps are disposable cap devices that contain an amount of cleaning or disinfecting solution for sterilizing portions of the port, hub, and valve.

Even with the use of disinfecting caps as part of catheter care maintenance, it is recognized that clinicians continue to seek out technologies and strategies to further reduce the risk of transmitting a CRBSI to a patient. Accordingly, many institutions have implemented processes with the goal of ensuring that all sterile practices and protocols are being followed. Such processes may track all aspects of catheter care, including when catheter lines are accessed and when a disinfecting cap is changed, as examples. However, it is recognized that existing processes are typically time consuming, duplicative and/or prone to error, leading to inefficiencies at the facility and lowering an overall standard of care.

Accordingly, it is desired to provide an automated system and method for providing catheter care and maintenance documentation, so as to reduce the amount of time required for such tasks and reduce errors and inefficiencies associated therewith. The automated system and method is able to track catheter care maintenance tasks, including when catheter lines are accessed and when a disinfecting cap change occurs. It is also desired to provide a detectable disinfecting cap that is useable with such a system and method, such that connection/disconnection and replacement of the disinfecting cap can be monitored.

### SUMMARY

According to a non-limiting embodiment or aspect, provided is a disinfecting cap engageable with a catheter connection. The disinfecting cap includes a housing defining an inner cavity, with the housing having a top wall, a multi-sided sidewall structure extending down from the top wall and including a plurality of sidewall sections, and an open bottom end. The disinfecting cap also includes a disinfecting member configured to disinfect portions of the catheter connection. Each of the plurality of sidewall sections includes a fiducial marker thereon, the fiducial marker containing a unique identifier for the disinfecting cap.

In some non-limiting embodiments or aspects, the fiducial marker is a marked data matrix containing the unique identifier.

In some non-limiting embodiments or aspects, the fiducial marker includes an AprilTag that provides position information for the disinfecting cap when detected by an associated image capture device, the position information comprising a three-dimensional (3D) position and orientation of the disinfecting cap relative to the image capture device.

In some non-limiting embodiments or aspects, the fiducial marker is formed directly on a respective sidewall section of the multi-sided sidewall structure via a laser marked UV laser, continuous inkjet, or IR technology.

In some non-limiting embodiments or aspects, the plurality of sidewall sections is between four and six sidewall sections.

In some non-limiting embodiments or aspects, the plurality of sidewall sections is five sidewall sections, such that the housing has a pentagonal geometry.

In some non-limiting embodiments or aspects, the housing is formed of a polymeric material that includes color additives configured to enhance contrast marking of the fiducial marker.

In some non-limiting embodiments or aspects, each of the sidewall sections includes a flat surface onto which the fiducial marker is applied.

In some non-limiting embodiments or aspects, the multi-sided sidewall structure includes rounded or tapered edges between each adjacent pair of sidewall sections.

In some non-limiting embodiments or aspects, the fiducial markers on the plurality of sidewall sections are spaced evenly about a central axis of the housing.

In some non-limiting embodiments or aspects, the multi-sided sidewall structure includes a cylindrical threaded inner surface configured to interlock with a mating feature of the catheter connector.

In some non-limiting embodiments or aspects, the housing comprises an outer housing, and wherein the disinfecting cap further comprises an inner cap member positioned within the outer housing, the inner cap member including a threaded inner surface configured to interlock with a mating feature of the catheter connector.

In some non-limiting embodiments or aspects, the inner cap member is joined to the outer housing via an adhesive, laser welding, or a snap feature.

According to another non-limiting embodiment or aspect, provided is a method of tracking a disinfecting cap as previously described. The method includes obtaining, with at least one processor, one or more images of a plurality of medical devices, captured by an image capture device. The method also includes determining, with the at least one processor, based on the one or more images, a presence of the disinfecting cap in the plurality of medical devices. The method further includes determining, with at least one processor, based on the one or more images, position information associated with a three-dimensional (3D) position of the disinfecting cap relative to the image capture device, when it is determined that the disinfecting cap is present in the plurality of medical devices. The method still further includes determining, with the at least one processor, based on the determined presence and/or the position information of the disinfecting cap, a status of the disinfecting cap.

In some non-limiting embodiments or aspects, determining the presence of the disinfecting cap in the plurality of medical devices includes checking, in the one or more images, for the unique identifier for the disinfecting cap.

In some non-limiting embodiments or aspects, determining the position information associated with the 3D position of the disinfecting cap relative to the image capture device includes determining the 3D position of the fiducial marker of the disinfecting cap relative to the image capture device.

In some non-limiting embodiments or aspects, the one or more images are a plurality of images captured over a period of time.

In some non-limiting embodiments or aspects, determining the status of the disinfecting cap comprises identifying, based on the plurality of images, a replacement of the disinfecting cap with a new disinfecting cap.

In some non-limiting embodiments or aspects, upon identifying replacement of the disinfecting cap with the new disinfecting cap, the method includes identifying, with the at least one processor, an IV line of the plurality of medical devices that was accessed by replacement of the disinfecting cap.

In some non-limiting embodiments or aspects, upon identifying the IV line that was accessed, the method includes providing, with the at least one processor, an alert to flush the IV line.

In some non-limiting embodiments or aspects, determining the status of the disinfecting cap comprises determining, based on the plurality of images, a dwell time of the disinfecting cap, and providing, with the at least one processor, an alert to change the disinfecting cap.

According to another non-limiting embodiment or aspect, provided is a system for tracking a disinfecting cap as previously described. The system includes an image capture device configured to capture images of an environment including a plurality of medical devices and at least one processor programmed and/or configured to obtain one or more images of the plurality of medical devices captured by the image capture device, determine based on the one or more images a presence of the disinfecting cap in the plurality of medical devices, determine based on the one or more images position information associated with a three-dimensional (3D) position of the disinfecting cap relative to the image capture device when it is determined that the disinfecting cap is present in the plurality of medical devices, and determine based on the determined presence and/or the position information of the disinfecting cap a status of the disinfecting cap.

### BRIEF DESCRIPTION OF THE DRAWINGS

Additional advantages and details are explained in greater detail below with reference to the exemplary embodiments that are illustrated in the accompanying schematic figures, in which:

FIG. 1A is a diagram of non-limiting embodiments or aspects of an environment in which systems, devices, products, apparatus, and/or methods, described herein, can be implemented;

FIG. 1B is a diagram of non-limiting embodiments or aspects of an implementation of an environment in which systems, devices, products, apparatus, and/or methods, described herein, can be implemented;

FIG. 2 is a diagram of non-limiting embodiments or aspects of components of one or more devices and/or one or more systems of FIGS. 1A and 1B;

FIG. 3A is a perspective view of implementations of non-limiting embodiments or aspects of medical devices;

FIG. 3B is a perspective view of the implementations of non-limiting embodiments or aspects of medical devices in FIG. 3A connected together;

FIG. 4A is a front top perspective view of a disinfecting cap in accordance with a non-limiting embodiment or aspect;

FIG. 4B is a front bottom perspective view of the disinfecting cap of FIG. 4A;

FIG. 4C is an exploded perspective view of the disinfecting cap of FIG. 4A;

FIG. 5A is a front top perspective view of a disinfecting cap in accordance with a non-limiting embodiment or aspect;

FIG. 5B is a front bottom perspective view of the disinfecting cap of FIG. 5A;

FIG. 5C is an exploded perspective view of the disinfecting cap of FIG. 5A; and

FIG. 6 is a flow chart of non-limiting embodiments or aspects of a process for tracking a disinfecting cap.

### DETAILED DESCRIPTION

It is to be understood that the present disclosure may assume various alternative configurations and variations, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

For purposes of the description hereinafter, the terms “end,” “upper,” “lower,” “right,” “left,” “vertical,” “horizontal,” “top,” “bottom,” “lateral,” “longitudinal,” and derivatives thereof shall relate to embodiments or aspects as they are oriented in the drawing figures. However, it is to be understood that embodiments or aspects may assume various alternative variations and step sequences, except where expressly specified to the contrary. It is also to be understood that the specific devices and processes illustrated in the attached drawings, and described in the following specification, are simply non-limiting exemplary embodiments or aspects. Hence, specific dimensions and other physical characteristics related to the embodiments or aspects of the embodiments or aspects disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and communicates the processed information to the second unit. In some non-limiting embodiments or aspects, a message may refer to a network packet (e.g., a data packet and/or the like) that includes data. It will be appreciated that numerous other arrangements are possible.

As used herein, the term “computing device” may refer to one or more electronic devices that are configured to directly or indirectly communicate with or over one or more networks. A computing device may be a mobile or portable computing device, a desktop computer, a server, and/or the like. Furthermore, the term “computer” may refer to any computing device that includes the necessary components to receive, process, and output data, and normally includes a display, a processor, a memory, an input device, and a network interface. A “computing system” may include one or more computing devices or computers. An “application” or “application program interface” (API) refers to computer code or other data sorted on a computer-readable medium that may be executed by a processor to facilitate the interaction between software components, such as a client-side front-end and/or server-side back-end for receiving data from the client. An “interface” refers to a generated display, such as one or more graphical user interfaces (GUIs) with which a user may interact, either directly or indirectly (e.g., through a keyboard, mouse, touchscreen, etc.). Further, multiple computers, e.g., servers, or other computerized devices directly or indirectly communicating in the network environment may constitute a “system” or a “computing system”.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

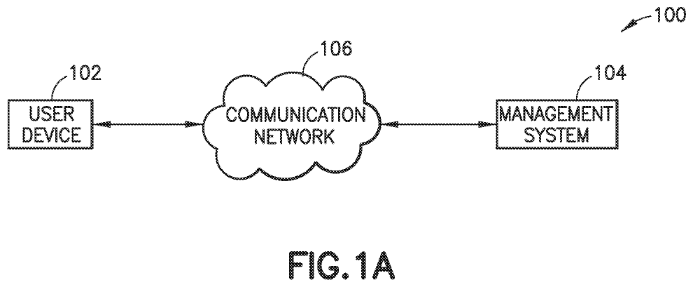

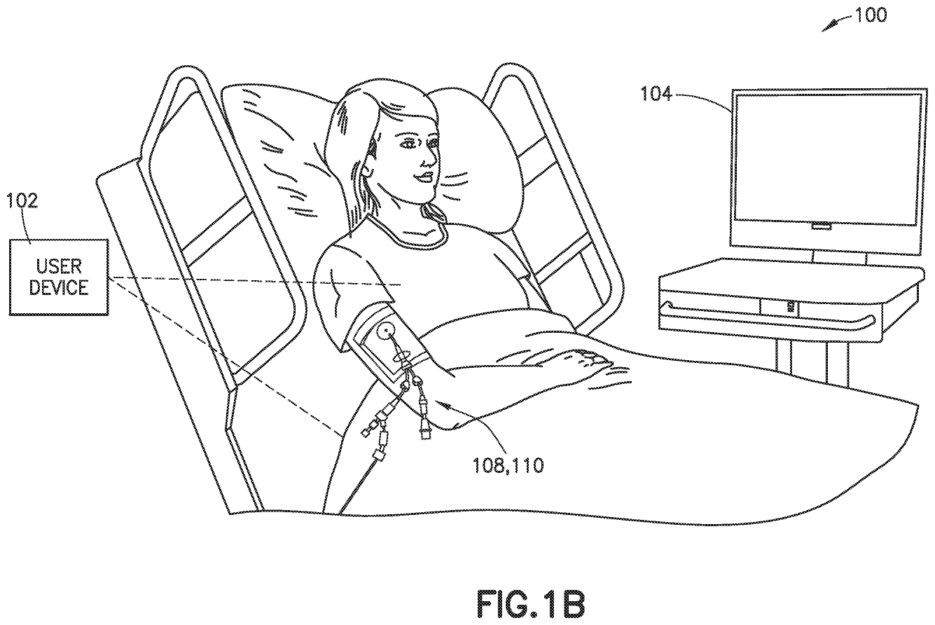

Referring now to FIG. 1A, FIG. 1A is a diagram of an example environment 100 in which devices, systems, methods, and/or products described herein, may be implemented. As shown in FIG. 1A, environment 100 includes user device 102, management system 104, and/or communication network 106. Systems and/or devices of environment 100 can interconnect via wired connections, wireless connections, or a combination of wired and wireless connections.

Referring also to FIG. 1B, FIG. 1B is a diagram of non-limiting embodiments or aspects of an implementation of environment 100 in which systems, devices, products, apparatus, and/or methods, described herein, can be implemented. For example, as shown in FIG. 1B, environment 100 may include a hospital room including a patient, one or more medical devices 108, one or more fiducial markers 110 associated with the one or more medical devices 108, and/or a caretaker (e.g., a nurse, etc.). While the medical devices 108 shown in FIG. 1B are provided as devices included in an IV catheter assembly, it is recognized that the medical devices 108 could be any of a number of medical devices associated with care or treatment of a patient.

User device 102 may include one or more devices capable of receiving information and/or data from management system 104 (e.g., via communication network 106, etc.) and/or communicating information and/or data to management system 104 (e.g., via communication network 106, etc.). For example, user device 102 may include one or more computing systems including one or more processors (e.g., one or more computing devices, one or more server computers, one or more mobile computing devices, one or more tablet computers, etc.). In some non-limiting embodiments or aspects, user device 102 may include a tablet computer or mobile computing device, such as an Apple® iPad, an Apple® iPhone, an Android® tablet, an Android® phone, and/or the like.

User device 102 may include one or more image capture devices (e.g., one or more cameras, one or more sensors, etc.) configured to capture one or more images of an environment (e.g., environment 100, etc.) surrounding the one or more image capture devices. For example, user device 102 may include one or more image capture devices configured to capture one or more images of the one or more medical devices 108, the one or more fiducial markers 110 associated with the one or more medical devices 108, and/or the patient. As an example, user device 102 may include at least one of the following image capture devices: a camera, a stereo camera, a LiDAR sensor, or any combination thereof.

Management system 104 may include one or more devices capable of receiving information and/or data from user device 102 (e.g., via communication network 106, etc.) and/or communicating information and/or data to user device 102 (e.g., via communication network 106, etc.). For example, management system 104 may include one or more computing systems including one or more processors (e.g., one or more computing devices, one or more server computers, one or more mobile computing devices, etc.). In some non-limiting embodiments or aspects, management system 104 includes and/or is accessible via a nurse station or terminal in a hospital. For example, management system 104 may provide bedside nurse support, nursing station manager support, retrospective reporting for nursing administration, and/or the like.

Communication network 106 may include one or more wired and/or wireless networks. For example, communication network 106 may include a cellular network (e.g., a long-term evolution (LTE) network, a third generation (3G) network, a fourth generation (4G) network, a fifth generation (5G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or a combination of these or other types of networks.

The number and arrangement of systems and devices shown in FIGS. 1A and 1B are provided as an example. There can be additional systems and/or devices, fewer systems and/or devices, different systems and/or devices, or differently arranged systems and/or devices than those shown in FIGS. 1A and 1B. Furthermore, two or more systems or devices shown in FIGS. 1A and 1B can be implemented within a single system or a single device, or a single system or a single device shown in FIGS. 1A and 1B can be implemented as multiple, distributed systems or devices. Additionally, or alternatively, a set of systems or a set of devices (e.g., one or more systems, one or more devices, etc.) of environment 100 can perform one or more functions described as being performed by another set of systems or another set of devices of environment 100.

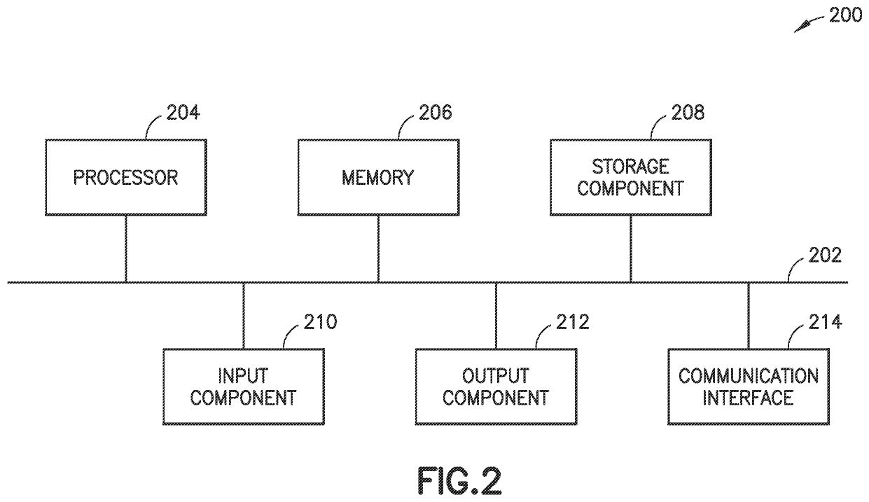

Referring now to FIG. 2, FIG. 2 is a diagram of example components of a device 200. Device 200 may correspond to user device 102 (e.g., one or more devices of a system of user device 102, etc.) and/or one or more devices of management system 104. In some non-limiting embodiments or aspects, user device 102 (e.g., one or more devices of a system of user device 102, etc.) and/or one or more devices of management system 104 may include at least one device 200 and/or at least one component of device 200. As shown in FIG. 2, device 200 may include bus 202, processor 204, memory 206, storage component 208, input component 210, output component 212, and communication interface 214.

Bus 202 may include a component that permits communication among the components of device 200. In some non-limiting embodiments or aspects, processor 204 may be implemented in hardware, software, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 206 may include random access memory (RAM), read-only memory (ROM), and/or another type of dynamic or static storage device (e.g., flash memory, magnetic memory, optical memory, etc.) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touch screen display, a keyboard, a keypad, a mouse, a button, a switch, a microphone, etc.). Additionally or alternatively, input component 210 may include a sensor for sensing information (e.g., a global positioning system (GPS) component, an accelerometer, a gyroscope, an actuator, etc.). Output component 212 may include a component that provides output information from device 200 (e.g., a display, a speaker, one or more light-emitting diodes (LEDs), etc.).

Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally or alternatively, hardwired circuitry may be used in place of or in combination with software instructions to perform one or more processes described herein. Thus, embodiments or aspects described herein are not limited to any specific combination of hardware circuitry and software.

Memory 206 and/or storage component 208 may include data storage or one or more data structures (e.g., a database, etc.). Device 200 may be capable of receiving information from, storing information in, communicating information to, or searching information stored in the data storage or one or more data structures in memory 206 and/or storage component 208.

The number and arrangement of components shown in FIG. 2 are provided as an example. In some non-limiting embodiments or aspects, device 200 may include additional components, fewer components, different components, or differently arranged components than those shown in FIG. 2. Additionally or alternatively, a set of components (e.g., one or more components) of device 200 may perform one or more functions described as being performed by another set of components of device 200.

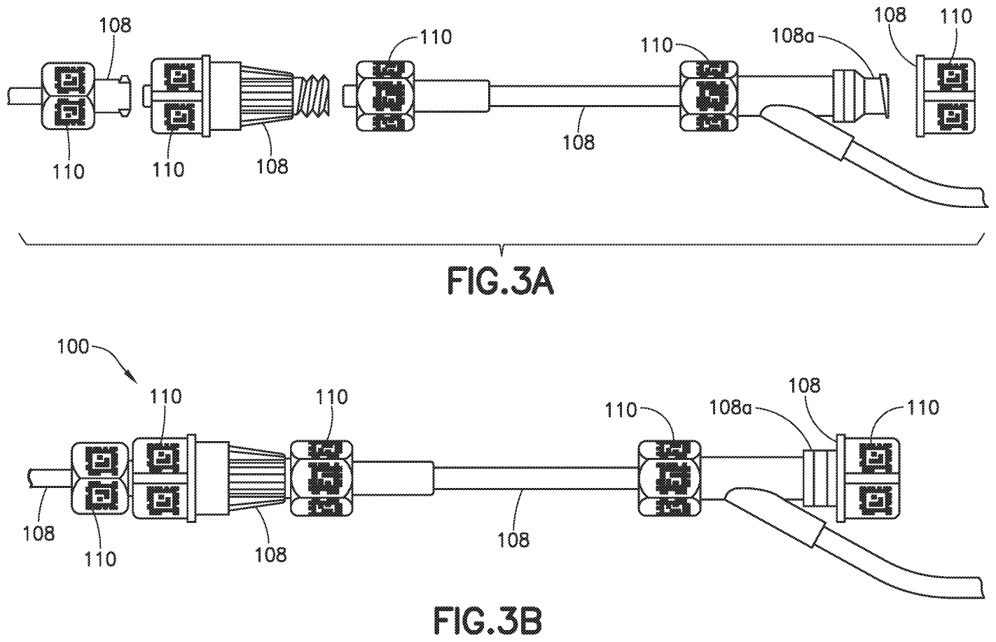

With reference again to FIG. 1B and the environment 100 shown therein that includes medical devices 108 and fiducial markers 110 associated with the care of a patient, it is recognized that the medical devices 108 may enter environment 100 (e.g., via the caretaker, etc.), may remain in environment 100 for a period of time (or indefinitely) during which the medical devices 108 may move within environment 100, may interact with (e.g., connect to, disconnect from, etc.) one or more other medical devices 108, the patient, and/or the caretaker, and/or may exit environment 100 at a subsequent time after entering environment 100 (e.g., via the caretaker, etc.). The medical devices 108 may include disposable medical devices or reusable medical devices. For example, the medical devices 108 may include at least one of the following types of medical devices a peripheral IV catheter (PIVC), a peripherally inserted central catheter (PICC), a midline catheter, a central venous catheter (CVC), a needleless connector, a catheter dressing, a catheter stabilization device, a disinfecting cap, an IV tubing set, an extension set, a Y connector, a stopcock, an infusion pump, a flush syringe, a medication delivery syringe, an IV fluid bag, a lumen adapter (e.g., a number of lumen adapters associated with a catheter may indicate a number of lumens included in the catheter, etc.), or any combination thereof. FIGS. 3A and 3B illustrate an arrangement of medical devices 108 (in a disconnected state (FIG. 3A) and a connected state (FIG. 3B)), according to a non-limiting embodiment, with each of the medical devices 108 including one or more fiducial markers 110 thereon. The medical devices 108 may collectively define an IV catheter assembly by which fluids may be administered to the patient or by which fluids may be withdrawn from the patient (i.e., blood draw).

A fiducial marker 110 (e.g., a tag, a label, a code, etc.) may be associated with (e.g., removably attached to, permanently attached to, integrated with, implemented on, etc.) a medical device 108. In some non-limiting embodiments or aspects, each medical device 108 in environment 100 may be associated with a fiducial marker 110. In some non-limiting embodiments or aspects, only a portion the medical devices 108 in environment 100 may be associated with fiducial markers 110.

A fiducial marker 110 may encapsulate an identifier associated with a type of a medical device 108 associated with the fiducial marker 110 and/or uniquely identify the medical device 108 associated with the fiducial marker 110 from other medical devices. For example, a fiducial marker 110 may encapsulate an identifier associated with at least one of the following types of medical devices: a peripheral IV catheter (PIVC), a peripherally inserted central catheter (PICC), a midline catheter, a central venous catheter (CVC), a needleless connector, a disinfecting cap, a disinfectant swab or wipe, an IV tubing set, an extension set, a Y connector, a stopcock, an infusion pump, a flush syringe, a medication delivery syringe, an IV fluid bag, or any combination thereof, and/or uniquely identify a medical device 108 (e.g., a first needless connector, disinfecting cap, etc.) from other medical devices (e.g., a second needless connector, etc.), including identifiers associated with a same type of medical device.

A fiducial marker 110 may encapsulate pose information associated with a 3D position of the fiducial marker 110. For example, fiducial marker 110 may include markings that, when captured in an image, enable computing a precise 3D position of the fiducial marker with respect to the image capture device that captured the image (e.g., an x, y, z coordinate position of the fiducial marker, etc.) and/or a precise 2D position of the fiducial marker in the image itself (e.g., a x, y coordinate positon of the fiducial marker in the image, etc.).

In some non-limiting embodiments or aspects, a fiducial marker 110 may include an AprilTag. For example, a fiducial marker 110 may include an AprilTag V3 of type customTag 48h12, which enables using AprilTag V3 detection to determine a unique ID, which may indicate a type of the medical device 108 associated with the fiducial marker (e.g., in leading digits, etc.) and/or a unique serial number for that specific medical device 108 (e.g., in the trailing digits, etc.), and/or a location (e.g., x, y, and z coordinates, directional vectors for Z, Y, and X axes, etc.) of the fiducial marker 110 in a field-of-view (FOV) of an image capture device. However, non-limiting embodiments or aspects are not limited thereto, and a fiducial marker 110 may include a QR code, a barcode (e.g., a 1D barcode, a 2D barcode, etc.), an Aztec code, a Data Matrix code, an ArUco marker, a colored pattern, a reflective pattern, a fluorescent pattern, a predetermined shape and/or color (e.g., a red pentagon, a blue hexagon, etc.), an LED pattern, a hologram, and/or the like that encapsulates an identifier associated with a type of a medical device 108 associated with the fiducial marker 110, uniquely identifies the medical device 108 associated with the fiducial marker 110 from other medical devices, and/or encapsulates pose information associated with a 3D position of the fiducial marker 110.

In some non-limiting embodiments or aspects, a fiducial marker 110 may include color calibration areas positioned adjacent to variable color regions to calibrate color in a wider range of lighting conditions. For example, for a 2×2 grid, a cell (1,1) in an upper-left corner of the gird may include a predetermined and/or standard calibration color region (e.g., neutral gray, etc.), and user device 102 and/or management system 104 may use the predetermined and/or standard calibration color region to calibrate colors in images used to detect or determine the fiducial marker 110 in those images and/or to detect or determine color changes in tissue of a patient (e.g., patient tissue adjacent an insertion site, etc.) in those images. In such an example, user device 102 and/or management system 104 may use the predetermined and/or standard calibration color region to orient the fiducial marker 110 to determine how to properly rotate and decode the colors in the fiducial marker 110 to decode the identifier encapsulated by the fiducial marker 110 and/or track the fiducial marker 110 within environment 100.

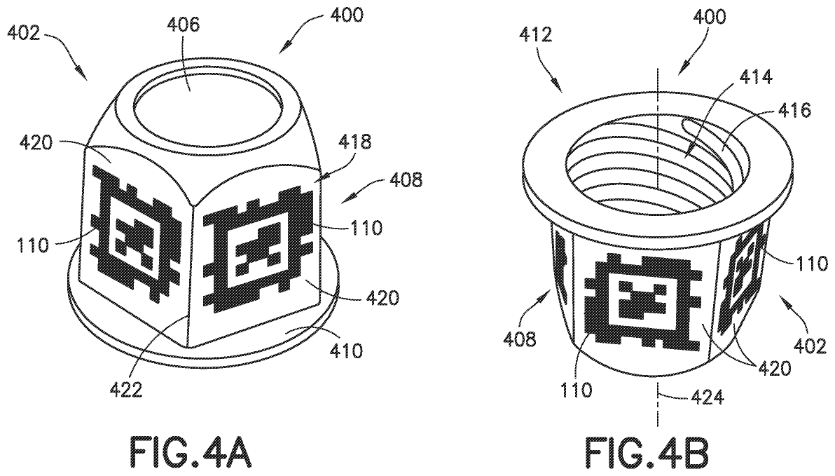

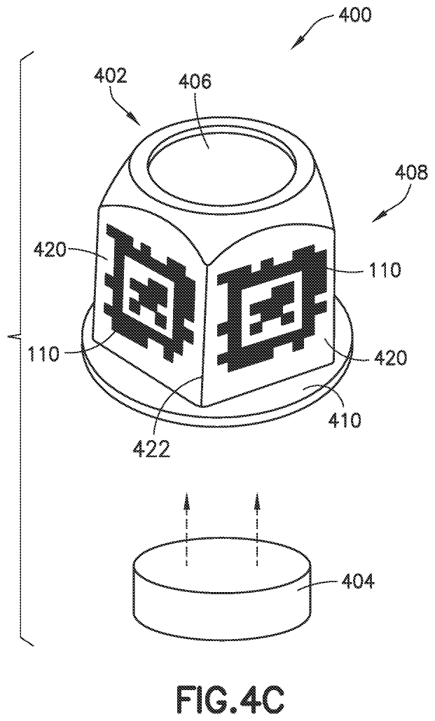

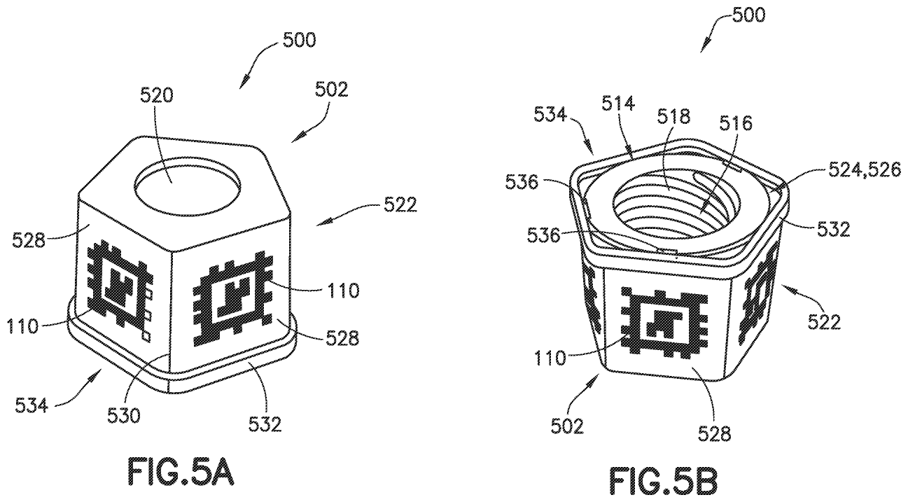

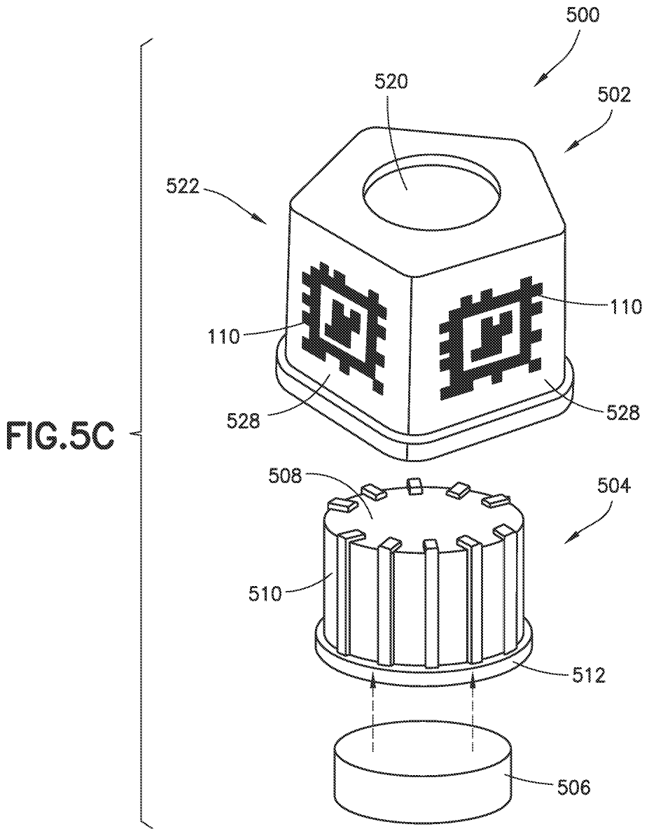

An exemplary medical device 108 that includes fiducial markers 110 thereon is shown in FIGS. 4 and 5 in accordance with aspects of the disclosure, with various embodiments of a disinfecting cap 400, 500 illustrated therein. As will be explained in further detail below, inclusion of fiducial markers 110 on the disinfecting caps 400, 500 of FIGS. 4 and 5 provides for monitoring of the positioning, usage and/or status of the disinfecting caps, including the removal/replacement thereof from a needleless access connector (NAC) on an IV line, as an example.

Referring first to FIGS. 4A-4C, a disinfecting cap 400 is shown in accordance with one aspect of the disclosure. The disinfecting cap 400 is generally configured to mate with, for example, a luer connection (e.g., female luer connection) of a needleless access connector (NAC) 108a (see FIGS. 3A and 3B) provided as part of an IV catheter assembly, as generally illustrated in FIGS. 3A and 3B. As one example, the disinfecting cap 400 is configured to mate with a SmartSite® NAC by Becton Dickinson. The disinfecting cap 400 includes a housing 402 within which is positioned an absorbent disinfecting member 404 having a disinfecting solution contained therein that is configured to disinfect portions of the NAC 108a. That is, disinfecting member 404 is configured to disinfect surfaces of the NAC 108a, including a threaded outer surface of the female luer connection thereon, so as prevent microbial ingress and minimize chances of CRBSI.

As shown in FIGS. 4A-4C, the housing 402 of disinfecting cap 400 may generally include a top wall 406, a sidewall structure 408, and a bottom flange 410 that defines an open bottom end 412 of the housing. The housing 402 is configured such that the top wall 406 and sidewall structure 408 collectively define or form an inner cavity 414 of the housing 402, with the open bottom end 412 of the housing 402 presenting an opening to the cavity 414 that provides for positioning of a female luer connection of NAC 108a within the cavity 414. The housing 402 may be formed as a single, unitary structure that may be easily manufactured. For example, the housing 402 can be formed from a thermoplastic polymer material, such as polyester, polycarbonate, polypropylene, polyethylene, polyethylene terephthalate, or acrylonitrile butadiene styrene, such that the housing 402 may be formed via an injection molding process, according to some embodiments.

The sidewall structure 408 of the housing 402 includes an inner surface 416 that is cylindrical in shape, so as to provide for receiving of the female luer connection of the NAC 108a therein. The inner surface 416 is configured as a threaded inner surface that engages and interlocks with a threaded outer surface or segment of the female luer connection of NAC 108a. In order to secure the disinfecting cap 400 to the NAC, the disinfecting cap 400 is twisted relative to the NAC 108a, so as to cause the corresponding threaded surfaces to engage and draw the NAC 108a together with the disinfecting cap 400.

The disinfecting member 404 is positioned within cavity 414 and is held in an upper portion of the cavity 414, such that the disinfecting member 404 may be positioned adjacent the top wall 406 of the housing 402 and extend down to fill a portion of the cavity 414. In some embodiments, the disinfecting member 404 may be held in position within the cavity 414 via contact thereof with the threaded inner surface 416 of the sidewall structure 408, with the threads on sidewall structure 408 keeping the disinfecting member 404 under radial compression. The disinfecting member 404 can be formed from an absorbent material, such as a porous foam (e.g., an open cell foam) or sponge, capable of absorbing a cleaning or disinfecting solution for cleaning and/or disinfecting portions of the NAC. The disinfecting member 404 can be provided (i.e., presoaked) with the cleaning or disinfecting solution, which may be an antimicrobial, anti-fungal, antibacterial, or antiviral solution that sterilizes surfaces of the NAC 108a. In some examples, the cleaning solution can be isopropyl alcohol (IPA), such as about 70% IPA. In other examples, the cleaning solution can be about 0.5% to about 3.5% chlorhexidine gluconate in combination with about 70% IPA. As the disinfecting member 404 is formed from a porous foam, sponge, or other elastomeric material, the disinfecting member 404 may be configured as compressive member. Accordingly, when the disinfecting cap 400 is mated with the female connection of the NAC 108a, the NAC 108a may compress the disinfecting member 404 when drawn into the cavity 414 via mating of the threaded outer surface of NAC 108a with the threaded inner surface disinfecting member 416 of housing 402. Compression of the disinfecting member 404 releases cleaning or disinfecting solution from the disinfecting member 404, with it being recognized that the amount of disinfecting solution released from the disinfecting member 404 may be controlled based on how much the disinfecting member 404 is compressed (based on an initial height/thickness of the disinfecting member 404) and the amount of cleaning or disinfecting solution contained therein. The cleaning or disinfecting solution contacts portions of the NAC 108a for disinfecting thereof.

According to aspects of the disclosure, an outer surface 418 of the sidewall structure 408 of the housing 402 is formed as a multi-sided sidewall structure including a plurality of sidewall portions 420. In the illustrated embodiment, multi-sided sidewall structure 408 includes five (5) sidewall portions 420 that collectively define a pentagonal sidewall structure 408; however, it is recognized that multi-sided sidewall structure 408 could alternatively include a lesser or greater number of sidewall portions 420, such as four sidewall portions 420 (square sidewall structure) or six sidewall portions 420 (hexagonal sidewall structure), as non-limiting examples. Each of the sidewall portions 420 may have tapered or rounded side edges 422, so as to provide a smooth transition between adjacent sidewall portions 420. Additionally, a top section of each sidewall portion 420 may have an arced profile, with the top wall 406 of housing 402 transitioning down to the sidewall portions 420 in a sloped fashion.

As shown in FIGS. 4A-4C, each of sidewall portions 420 includes a fiducial marker 110 thereon. As previously described, the fiducial marker 110 may be provided in any of a number of suitable formats, an AprilTag, a QR code, a barcode (e.g., a 2D barcode), an Aztec code, a Data Matrix code, an ArUco marker, a colored pattern, a reflective pattern, a fluorescent pattern, a predetermined shape and/or color (e.g., a red pentagon, a blue hexagon, etc.), an LED pattern, a hologram, and/or the like. In the illustrated embodiment, each of the fiducial markers 110 is provided as a 2D detectable data matrix that is directly marked on the housing 402, such as via a laser marked UV laser, continuous inkjet, or IR technology. In some embodiments, color additives may be added to the polymeric material from which housing 402 is formed to enhance contrast marking of the fiducial markers 110. The fiducial markers 110 are marked on sidewall portions 420 so as to be arranged symmetrically and spaced evenly about a central axis 424 of the disinfecting cap 400, which may enable at least one fiducial marker 110 being presented to the FOV of an image capture device 102 (FIGS. 1A and 1B) regardless of an orientation of the disinfecting cap 400, so as to enhance marker recognition and ease of use.

Referring now to FIGS. 5A-5C, a disinfecting cap 500 is shown in accordance with another aspect of the disclosure. As with disinfecting cap 400, the disinfecting cap 500 is generally configured to mate with a luer connection (e.g., female luer connection) of a NAC 108a, such as a SmartSite® NAC by Becton Dickinson for example. The disinfecting cap 500 is configured to disinfect portions of the NAC 108a (e.g., a threaded outer surface of the female luer connection thereon), so as prevent microbial ingress and minimize chances of CRBSI.

As shown in FIGS. 5A-5C, the disinfecting cap 500 includes an outer cap housing 502, an inner cap member 504, and an absorbent disinfecting member 506 having a disinfecting solution contained therein. The inner cap member 504 and absorbent disinfecting member 506 may be provided as a standard-type cap, with the inner cap member 504 provided as a generally cylindrical member that includes a top wall 508, a sidewall 510, and a bottom flange 512 that defines an open bottom end 514 of the inner cap member 504. The inner cap member 504 is configured such that the top wall 508 and sidewall 510 collectively define or form an inner cavity 516 of the inner cap member 504 within which the disinfecting member 506 is positioned, with the open bottom end 514 of the inner cap member 504 presenting an opening that provides for positioning of a female luer connection of NAC 108a therein. The sidewall 510 of the inner cap member 504 includes a threaded inner surface 518 that engages and interlocks with a threaded outer surface or segment of the female luer connection of NAC 108a.

The disinfecting member 506 is positioned within cavity 516 and is held in an upper portion of the cavity 516, such that the disinfecting member 506 may be positioned adjacent the top wall 508 of the inner cap member 504 and extend down to fill a portion of the cavity 516. As previously described, the disinfecting member 506 can be formed from an absorbent material, such as a porous foam (e.g., an open cell foam) or sponge, capable of absorbing a cleaning or disinfecting solution for cleaning and/or disinfecting portions of the NAC 108a. The disinfecting member 506 can be provided (i.e., presoaked) with the cleaning or disinfecting solution, which may be an antimicrobial, anti-fungal, antibacterial, or antiviral solution that sterilizes surfaces of the NAC 108a. When the disinfecting cap 500 is mated with the female connection of the NAC 108a, the NAC 108a may compress the disinfecting member 506, which releases cleaning or disinfecting solution from the disinfecting member 506 for disinfecting the NAC 108a.

According to aspects of the disclosure, the outer cap housing 502 is provided as a separate component from inner cap member 504 and may be joined to the inner cap member 504 so as to be positioned thereover. The outer cap housing 502 includes a top wall 520 and a multi-sided sidewall structure 522 that collectively define a cavity 524 having an open bottom end 526. In the illustrated embodiment, the multi-sided sidewall structure 522 includes five (5) sidewall portions 528 that collectively define a pentagonal sidewall structure 522; however, it is recognized that multi-sided sidewall structure 522 could alternatively include a lesser or greater number of sidewall portions 528, such as four sidewall portions 528 (square sidewall structure) or six sidewall portions 528 (hexagonal sidewall structure), as non-limiting examples. Each of the sidewall portions 528 may have tapered or rounded side edges 530, so as to provide a smooth transition between adjacent sidewall portions 528.

As shown in FIGS. 5A-5C, the outer cap housing 502 is constructed (i.e., based on a height and perimeter length of the multi-sided sidewall structure 522) such that the cavity 524 defined thereby is sufficiently sized to receive the inner cap member 504 therein. In some embodiments, the cavity 524 is sized such that the inner cap member 504 is positioned within the outer cap housing 502 in a manner that causes the bottom flange 512 of inner cap member 504 to be seated against a lip portion 532 of multi-sided sidewall structure 522, and with the bottom flange 512 flush with a bottom edge 534 of the multi-sided sidewall structure 522. According to embodiments, the inner cap member 504 may be secured within outer cap housing 502 in a permanent fashion or in a fashion that provides for selective removal of the outer cap housing 502 from the inner cap member 504. In embodiments where the inner cap member 504 and outer cap housing 502 are permanently secured to one another, the outer cap housing 502 may be glued or laser welded to the inner cap member 504. In embodiments where the outer cap housing 502 may be selectively removed from the inner cap member 504, the outer cap housing 502 and inner cap member 504 may couple together via one or more snap features 536 formed on the lip portion 532 and/or an inner surface of the multi-wall sidewall structure 522.

As previously described with respect to disinfecting cap 400, each of the sidewall portions 528 of multi-wall sidewall structure 522 on outer cap housing 502 may include a fiducial marker 110 thereon, with the illustrated embodiment showing that each of the fiducial markers 110 is provided as a 2D detectable data matrix that is directly marked on the outer cap housing 502, such as via a laser marked UV laser, continuous inkjet, or IR technology. The fiducial markers 110 are marked on sidewall portions 528 so as to be arranged symmetrically and spaced evenly about a central axis 538 of the disinfecting cap 500, which may enable at least one fiducial marker 110 being presented to the FOV of an image capture device 102 (FIGS. 1A and 1B) regardless of an orientation of the disinfecting cap 500, so as to enhance marker recognition and ease of use.

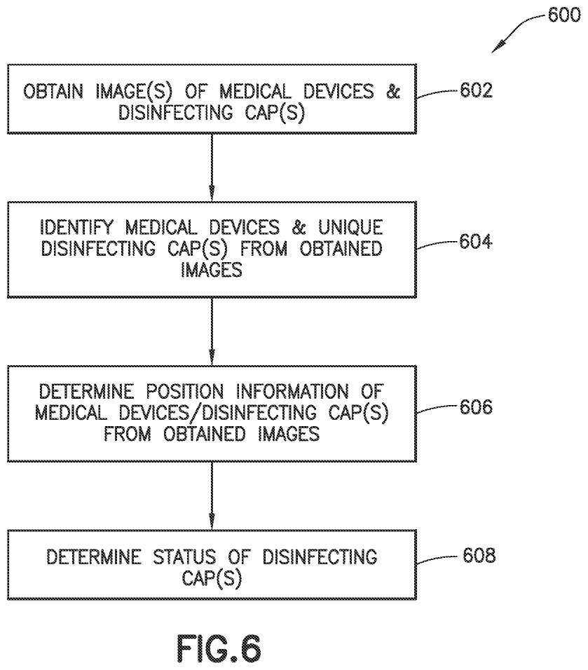

Referring now to FIG. 6, a flowchart of a method 600 for vascular access management is shown according to a non-limiting embodiment or aspect of the disclosure. In particular, the method 600 is directed to tracking a disinfecting cap 400, 500 as shown and described in FIGS. 4 and 5. In some non-limiting embodiments or aspects, one or more of the steps of process 400 may be performed (e.g., completely, partially, etc.) by user device 102 (e.g., one or more devices of a system of user device 102, etc.). In some non-limiting embodiments or aspects, one or more of the steps of process 400 may be performed (e.g., completely, partially, etc.) by another device or a group of devices separate from or including user device 102, such as management system 104 (e.g., one or more devices of management system 104, etc.).

As shown in FIG. 6, at step 602, process 600 includes obtaining one or more images. For example, user device 102 (i.e., an image capture device thereof) may obtain image(s) (e.g., a single image, a plurality of images, a series of images, etc.) of a plurality of medical devices 108, such as may be included in an IV catheter assembly, including of one or more disinfecting caps 400, 500 provided on a NAC of the IV catheter assembly. As an example, an image capture device of user device 102 may capture the image(s) of the plurality of medical devices 108, including one or more disinfecting caps 400, 500. In such an example, a nurse may use user device 102 to take one or more images of a catheter site of a patient that includes the plurality of medical devices 108 (i.e., an IV catheter assembly), including one or more disinfecting caps 400, 500, such as generally shown in FIG. 1B. In some non-limiting embodiments or aspects, a nurse may take a series of images over a period of time, such as capturing one or more images of the plurality of medical devices 108 and disinfecting cap 400, 500 on each of a number of shifts worked by the nurse.

As shown in FIG. 6, at step 604, process 600 includes identifying a plurality of medical devices 108 of the IV catheter assembly, including determining the presence of a disinfecting cap 400, 500 in the medical devices. For example, user device 102 and/or management system 104 may determine, based on the images, the types of medical devices 108 within the environment over a period of time and/or specific medical devices within the environment, including identifying unique disinfecting cap(s) 400, 500 within the environment. In identifying the plurality of medical devices 108, the user device 102 may determine or identify, based on the image, the plurality of fiducial markers 110 associated with the medical devices 108. For example, the plurality of fiducial markers 110 may include a plurality of AprilTags, and user device 102 may process the image using AprilTag detection software to determine types of the medical devices 108 associated with the fiducial markers 110 and/or a unique serial number for the specific medical devices 108. With regard to disinfecting cap(s) 400, 500, the fiducial markers 110 thereon may include a plurality of AprilTags, and user device 102 may process the image using AprilTag detection software to identify a unique serial number for the disinfecting cap(s) 400, 500. Accordingly, at step 604, the presence of a particular disinfecting cap 400, 500 in the plurality of medical devices 108 may be quickly identified by the user device 102 and/or management system 104.

As shown in FIG. 6, at step 606, process 600 includes determining locations, trajectories, and/or orientations (i.e., “position information”) of the plurality of medical devices 108, including disinfecting cap(s) 400, 500, as well as locations of a plurality of medical devices 108 over the period of time, a plurality of trajectories of the plurality of medical devices 108 within the environment over the period of time, and/or a plurality of orientations of the plurality of medical devices 108 within the environment over the period of time. For example, user device 102 and/or management system 104 may determine, based on the image, position information associated with a 3D position of the plurality of medical devices 108 (including disinfecting cap(s) 400, 500) relative to the image capture device and/or a 2D position of the medical device 108 in the image itself. That is, user device 102 may determine the position information associated with the 3D position of the plurality of medical devices 108 relative to the image capture device and/or the 2D position of the plurality of medical devices 108 in the image itself by determining or identifying, based on the image, the plurality of fiducial markers 110 associated with the medical devices and the pose information associated with the 3D positions and/or 2D positions of the plurality of fiducial markers 110. For example, the plurality of fiducial markers 110 may include a plurality of AprilTags, and user device 102 may process the image using AprilTag detection software to compute a precise 3D position, orientation, and/or identity of the plurality of fiducial markers 110 relative to the image capture device that captured the image and/or a precise 2D position of the plurality of fiducial markers 110 in the image itself. For each medical device 108, the 3D position of the fiducial marker 110 associated with that medical device relative to the image capture device may include x, y, and z coordinates of the fiducial marker 110 and/or directional vectors for Z, Y, and X axes of the fiducial marker 110. In such an example, for each medical device 108 of the first group of medical devices, the 2D position of the fiducial marker 110 in the image itself may include x, y coordinates of the fiducial marker 110 in the image and/or direction vectors for Y and X axe of the fiducial marker.

As shown in FIG. 6, at step 608, process 600 includes determining a status of the disinfecting cap(s) 400, 500 present in the plurality of medical devices 108. For example, the user device 102 and/or management system 104 may determine, based on the determined presence of specific disinfecting cap(s) 400, 500 and/or the position information of the disinfecting cap(s) 400, 500, a status of the disinfecting cap(s) 400, 500. The status of the disinfecting cap(s) 400, 500 may encompass a number of different usages of the disinfecting cap(s) 400, 500 and their association with the other medical devices 108, including the connection/disconnection of disinfecting cap(s) 400, 500 from one or more medical devices 108 (i.e., NACs 108a), the replacement of a disinfecting cap 400, 500 with a new disinfecting cap 400, 500, and/or which IV lines in the IV catheter assembly have been accessed.

In one non-limiting embodiment or aspect, the user device 102 and/or management system 104 may compare a first image of the plurality of medical devices 108 and disinfecting cap(s) 400, 500 taken by a nurse during a first shift to a second image of the plurality of medical devices 108 and disinfecting cap(s) 400, 500 taken by the nurse during a second shift, so as to identify whether a disinfecting cap 400, 500 has been replaced with a new disinfecting cap 400, 500. In each image, the user device 102 and/or management system 104 may determine or identify a particular disinfecting cap 400, 500 in use in the IV catheter assembly via the identification of the fiducial marker 110 associated with a disinfecting cap 400, 500. Accordingly, if a particular disinfecting cap 400, 500 is identified in the first image (via its unique fiducial marker 110) and that same disinfecting cap 400, 500 is no longer present in the second image—but instead a new disinfecting cap 400, 500 is identified in the second image (via its unique fiducial marker 110) at the location at which the old disinfecting cap 400, 500 was identified—then the user device 102 and/or management system 104 may determine that a cap replacement was performed.

In one non-limiting embodiment or aspect, if the user device 102 and/or management system 104 has determined/identified that a disinfecting cap 400, 500 has been replaced with a new disinfecting cap 400, 500, the user device 102 and/or management system 104 may determine or identify an IV line (of the plurality of medical devices 108) that was accessed during replacement of the disinfecting cap 400, 500. In identifying an IV line that was accessed, the user device 102 may process the position information of the plurality of medical devices 108 and/or the types of the plurality of medical devices 108 to determine which IV line the old/new disinfecting caps 400, 500 were/are connected to. As previously described, using the AprilTags provided in the fiducial markers 110 on the medical devices 108, the user device 102 may process the image(s) using AprilTag detection software to compute a precise 3D position, orientation, and/or identity of the plurality of fiducial markers 110 relative to the image capture device that captured the image and/or a precise 2D position of the plurality of fiducial markers 110 in the image itself, thereby enabling association of the identified disinfecting cap 400, 500 with an IV line (and NAC 108a) to which it is connected.

In one non-limiting embodiment or aspect, in connection with identifying an IV line (of the plurality of medical devices 108) that was accessed by replacement of a disinfecting cap 400, 500, the user device 102 and/or management system 104 may further be configured or programmed to provide an alert or reminder to the nurse to flush that IV line. The alert may be a visual (e.g., textual) or audible alert output by the user device 102, with the alert enabling compliance with proper sterilization protocols.

In still other non-limiting embodiments or aspects, user device 102 and/or management system 104 may determine a dwell time associated with disinfecting cap(s) 400, 500—i.e., how long a same disinfecting cap 400, 500 has been connected to its associated NAC 108a—in recognition that disinfecting cap(s) 400, 500 may periodically need to be replaced. If the dwell time exceeds a pre-determined dwell time threshold, the user device 102 may provide an alert indicating that it is time to replace the disinfecting cap 400, 500 with a new disinfecting cap 400, 500.

In still other non-limiting embodiments or aspects, user device 102 and/or management system 104 may identify disinfecting cap(s) 400, 500 that have become disconnected from their associated medical device 108 (i.e., NAC). The user device 102 may process the position information of the plurality of medical devices 108 and the types of the plurality of medical devices 108, including the location and identify of disinfecting cap(s) 400, 500, to identify a disinfecting cap 400, 500 that has come disconnected and may be floating around on a bed or a patient close to other tagged objects, with the user device 102 automatically determining that a disinfecting cap 400, 500 is not connected to another medical device 108 if a distance between the cap and the other medical device exceeds a predetermined threshold distance.

In still other non-limiting embodiments or aspects, user device 102 and/or management system 104 may store each image acquired by the user device 102, so as to document site checks performed by the nurse. By documenting and recording such site checks, it may be quickly and efficiently confirmed that proper vascular access management is being performed, including that vascular access devices are being properly maintained and that disinfecting protocols and procedures are being followed.

Beneficially, embodiments of the invention thus provide a detectable disinfecting cap that may be implemented with a vascular access management system and method that enables tracking of the disinfecting cap and of tasks or procedures associated therewith. The disinfecting cap includes a plurality of fiducial marks spaced evenly about a central axis thereof that may be read/detected by the vascular access management system to uniquely identify the disinfecting cap and locate the disinfecting cap relative to an image capture device and relative to other medical devices. Via tracking of the detectable disinfecting cap, cap changes and the accessing of IV lines may be tracked, thereby enabling generation of reminders to flush the IV line, so as to reduce the likelihood of occluded lines. Capturing of images of the disinfecting cap and associated medical devices also allows for the creation of photo logs that can be reviewed over time, thereby enhancing documentation accuracy, VAM protocol compliance, and providing the ability to provide future training to improve care.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

What is claimed is:

1. A disinfecting cap engageable with a catheter connection, the disinfecting cap comprising:
a housing defining an inner cavity, the housing comprising:
a top wall;
a multi-sided sidewall structure extending down from the top wall, the multi-sided sidewall structure comprising a plurality of sidewall sections; and
an open bottom end; and
a disinfecting member configured to disinfect portions of the catheter connection;
wherein each of the plurality of sidewall sections includes a fiducial marker thereon, the fiducial marker containing a unique identifier for the disinfecting cap.

2. The cap of claim 1, wherein the fiducial marker comprises a marked data matrix containing the unique identifier.

3. The cap of claim 1, wherein the fiducial marker includes an AprilTag that provides position information for the disinfecting cap when detected by an associated image capture device, the position information comprising a three-dimensional (3D) position and orientation of the disinfecting cap relative to the image capture device.

4. The cap of claim 1, wherein the fiducial marker is formed directly on a respective sidewall section of the multi-sided sidewall structure via a laser marked UV laser, continuous inkjet, or IR technology.

5. The cap claim 1, wherein the plurality of sidewall sections comprises between four and six sidewall sections.

6. The cap of claim 5, wherein the plurality of sidewall sections comprises five sidewall sections, such that the housing has a pentagonal geometry.

7. The cap of claim 1, wherein the housing is formed of a polymeric material that includes color additives configured to enhance contrast marking of the fiducial marker.

8. The cap of claim 1, wherein each of the sidewall sections comprises a flat surface onto which the fiducial marker is applied.

9. The cap of claim 8, wherein the multi-sided sidewall structure comprises rounded or tapered edges between each adjacent pair of sidewall sections.

10. The cap of claim 1, wherein the fiducial markers on the plurality of sidewall sections are spaced evenly about a central axis of the housing.

11. The cap of claim 1, wherein the multi-sided sidewall structure includes a cylindrical threaded inner surface configured to interlock with a mating feature of the catheter connector.

12. The cap of claim 1, wherein the housing comprises an outer housing, and wherein the disinfecting cap further comprises an inner cap member positioned within the outer housing, the inner cap member including a threaded inner surface configured to interlock with a mating feature of the catheter connector.

13. The cap of claim 12, wherein the inner cap member is joined to the outer housing via an adhesive, laser welding, or a snap feature.

14. A method of tracking a disinfecting cap engageable with a catheter connection, method comprising:
providing a disinfecting cap comprising a housing defining an inner cavity, the housing comprising a top wall, a multi-sided sidewall structure extending from the top wall, the multi-sided sidewall structure comprising a plurality of sidewall sections, an open bottom end, and a disinfecting member configured to disinfect portions of the catheter connection, wherein each of the plurality of sidewall sections includes a fiducial marker thereon, the fiducial marker containing a unique identifier for the disinfecting cap;
obtaining, with at least one processor, one or more images of a plurality of medical devices, captured by an image capture device;
determining, with the at least one processor, based on the one or more images, a presence of the disinfecting cap in the plurality of medical devices;
determining, with at least one processor, based on the one or more images, position information associated with a three-dimensional (3D) position of the disinfecting cap relative to the image capture device, when it is determined that the disinfecting cap is present in the plurality of medical devices; and
determining, with the at least one processor, based on the determined presence and/or the position information of the disinfecting cap, a status of the disinfecting cap.

15. The method of claim 14, wherein determining the presence of the disinfecting cap in the plurality of medical devices includes checking, in the one or more images, for the unique identifier for the disinfecting cap.

16. The method of claim 15, wherein determining the position information associated with the 3D position of the disinfecting cap relative to the image capture device comprises determining the 3D position of the fiducial marker of the disinfecting cap relative to the image capture device.

17. The method of claim 14, wherein the one or more images comprises a plurality of images captured over a period of time.

18. The method of claim 17, wherein determining the status of the disinfecting cap comprises identifying, based on the plurality of images, a replacement of the disinfecting cap with a new disinfecting cap.

19. The method of claim 18, further comprising, upon identifying replacement of the disinfecting cap with the new disinfecting cap, identifying, with the at least one processor, an IV line of the plurality of medical devices that was accessed by replacement of the disinfecting cap.

20. The method of claim 19, further comprising, upon identifying the IV line that was accessed, providing, with the at least one processor, an alert to flush the IV line.

21. The method of claim 17, wherein determining the status of the disinfecting cap comprises determining, based on the plurality of images, a dwell time of the disinfecting cap, and wherein the method further comprises providing, with the at least one processor, an alert to change the disinfecting cap.

22. A system for tracking a disinfecting cap, comprising:
a disinfecting cap engageable with a catheter connection, the disinfecting cap comprising a housing defining an inner cavity, a top wall, a multi-sided sidewall structure extending from the top wall, the multi-sided sidewall structure comprising a plurality of sidewall sections, an open bottom end, and a disinfecting member configured to disinfect portions of the catheter connection, wherein each of the plurality of sidewall sections includes a fiducial marker thereon, the fiducial marker containing a unique identifier for the disinfecting cap;
an image capture device configured to capture images of an environment including a plurality of medical devices; and
at least one processor programmed and/or configured to:
obtain one or more images of the plurality of medical devices captured by the image capture device;
determine, based on the one or more images, a presence of the disinfecting cap in the plurality of medical devices;
when it is determined that the disinfecting cap is present in the plurality of medical devices, determine, based on the one or more images, position information associated with a three-dimensional (3D) position of the disinfecting cap relative to the image capture device; and determine, based on the determined presence and/or the position information of the disinfecting cap, a status of the disinfecting cap.
